(19) United States
(12) Patent Application Publication
(10) Pub. No.: US 2019/0175892 A1
(43) Pub. Date: Jun. 13, 2019

(54) SKIN MEDICINE DOSE CONTROL

(71) Applicant: INTERNATIONAL BUSINESS MACHINES CORPORATION, Armonk, NY (US)

(72) Inventors: Michael S. Gordon, Yorktown Heights, NY (US); James R. Kozloski, New Fairfield, CT (US); John S. Murdock, JR., New Fairfield, CT (US); Clifford A. Pickover, Yorktown Heights, NY (US)

(21) Appl. No.: 16/277,474

(22) Filed: Feb. 15, 2019

Related U.S. Application Data
(63) Continuation-in-part of application No. 15/093,219, filed on Apr. 7, 2016.

(51) Int. Cl. A61M 35/00

(52) U.S. Cl. CPC A61M 35/00; A61M 2205/3303; A61M 2230/50; A61M 2205/50; A61M 2210/04; A61M 2205/3306

(57) ABSTRACT

A method and system are provided for managing a skin condition. The method includes determining, using at least one of an image capture device and a temperature sensor, a skin inflammation value. The method further includes determining, by a hardware processor, a risk value associated with an improper determination of the skin inflammation value. The method also includes triggering, by the hardware processor, an action relating to the skin condition based on the skin inflammation value and the risk value.

### BACKGROUND

### Technical Field

The present invention generally relates to medicine dosing, and more particularly to skin medicine dose control.

### Description of the Related Art

Skin conditions are becoming ever prevalent and can become quite a nuisance to persons suffering from the skin conditions. Dosing control is a problem in addressing skin conditions, as the same depends on the current state of the condition and other factors. Thus, there is a need for a skin medicine dose control method that can provide the proper skin medicine dose control for a person suffering from and looking to cure and/or otherwise ameliorate the effects of a skin condition.

### SUMMARY

According to an aspect of the present principles, a method is provided for managing a skin condition. The method includes determining, using at least one of an image capture device and a temperature sensor, a skin inflammation value. The method further includes determining, by a hardware processor, a risk value associated with an improper determination of the skin inflammation value. The method also includes triggering, by the hardware processor, an action relating to the skin condition based on the skin inflammation value and the risk value.

According to another aspect of the present principles, a non-transitory computer readable storage medium is provided. The non-transitory computer readable storage medium includes a computer readable program for managing a skin condition. The computer readable program when executed on a computer causes the computer to perform steps of a method. The method includes determining, using at least one of an image capture device and a temperature sensor, a skin inflammation value. The method further includes determining, by a hardware processor, a risk value associated with an improper determination of the skin inflammation value. The method also includes triggering, by the hardware processor, an action relating to the skin condition based on the skin inflammation value and the risk value.

According to yet another aspect of the present principles, a system is provided. The system includes at least one of an image capture device and a temperature sensor for determining a skin inflammation value. The system further includes a hardware processor for determining a risk value associated with an improper determination of the skin inflammation value, and triggering an action relating to the skin condition based on the skin inflammation value and the risk value.

These and other features and advantages will become apparent from the following detailed description of illustrative embodiments thereof, which is to be read in connection with the accompanying drawings.

### BRIEF DESCRIPTION OF THE SEVERAL VIEWS OF THE DRAWINGS

The disclosure will provide details in the following description of preferred embodiments with reference to the following figures wherein:

FIG. 1 shows an exemplary processing system to which the present principles may be applied, in accordance with an embodiment of the present principles;

FIG. 2 shows a system for skin medicine dose control, in accordance with an embodiment of the present principle;

FIGS. 3-4 show a method for skin medicine dose control, in accordance with an embodiment of the present principles;

FIG. 5 shows another method for skin medicine dose control, in accordance with an embodiment of the present principles;

FIG. 6 shows an exemplary cloud computing node, in accordance with an embodiment of the present principles;

FIG. 7 shows an exemplary cloud computing environment, in accordance with an embodiment of the present principles; and

FIG. 8 shows exemplary abstraction model layers, in accordance with an embodiment of the present principles.

### DETAILED DESCRIPTION

The present principles are directed to skin medicine dose control.

In an embodiment, the present principles a method, apparatus, and service for management of skin conditions (problems). In an embodiment, the present principles involve (1) determining a skin inflammation value V (or related value), (2) determining a risk value R associated with improper determination of V, and then, based on V and R, (3) triggering a real-world action. The step of triggering a real-world action can include, but is not limited to, controlling the amount of medication that exits a medicine-application device and/or controlling the nature of sunscreen lotion (e.g., SPF factor/dynamic SPF module), and/or sending an alert to a healthcare professional, and/or tracking V and R through time (e.g., a photo log) and/or so forth. In an embodiment, in the case of an infection, a temperature sensor could be added to the medicine-application device so that an action is optionally taken or not taken if the temperature of the infected (e.g., inflamed) area increases beyond a predetermined threshold. Also, in an embodiment, one way to deter over-medication is to determine the usage pattern and frequency of use of the medicine-application device and then reduce the amount of medicine applied if a dosage per time exceeds a threshold.

The determining of the aforementioned risk parameter R may be based on patient information (e.g., do not increase the dose beyond a threshold if there is a possible error in the value of V and if the patient has been diagnosed with a particular condition).

A cognitive-computing element can also be used to ascertain a user's state with respect to itching, pain, embarrassment, forecast or current attendance at an important event, and so forth.

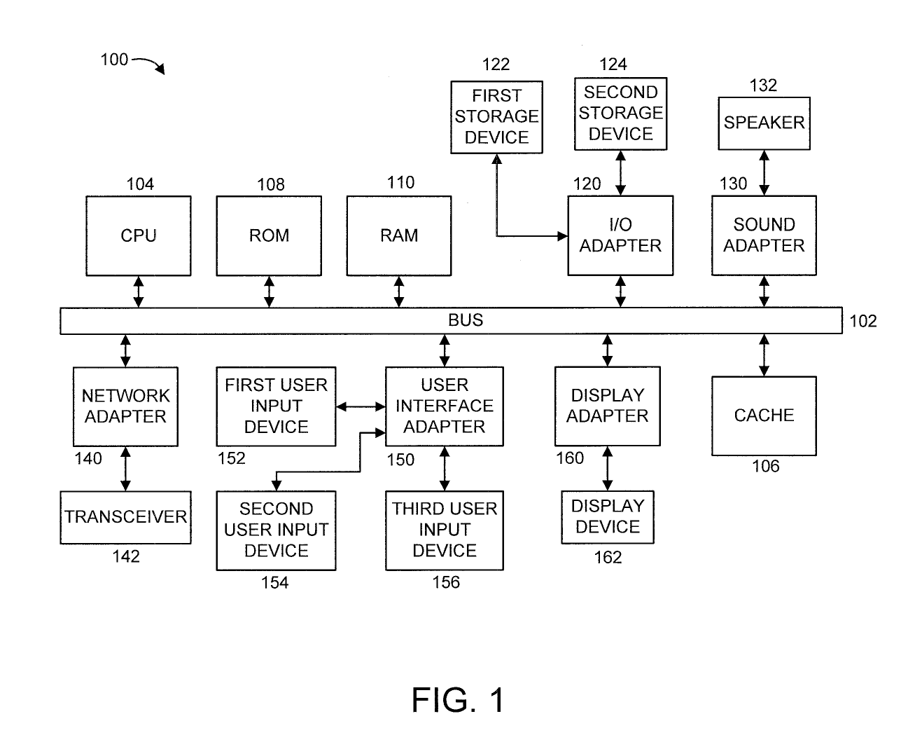

FIG. 1 shows an exemplary processing system 100 to which the present principles may be applied, in accordance with an embodiment of the present principles. The processing system 100 includes at least one processor (CPU) 104 operatively coupled to other components via a system bus 102. A cache 106, a Read Only Memory (ROM) 108, a Random Access Memory (RAM) 110, an input/output (I/O) adapter 120, a sound adapter 130, a network adapter 140, a user interface adapter 150, and a display adapter 160, are operatively coupled to the system bus 102.

A first storage device 122 and a second storage device 124 are operatively coupled to system bus 102 by the I/O adapter 120. The storage devices 122 and 124 can be any of a disk storage device (e.g., a magnetic or optical disk storage device), a solid state magnetic device, and so forth. The storage devices 122 and 124 can be the same type of storage device or different types of storage devices.

A speaker 132 is operatively coupled to system bus 102 by the sound adapter 130. A transceiver 142 is operatively coupled to system bus 102 by network adapter 140. A display device 162 is operatively coupled to system bus 102 by display adapter 160.

A first user input device 152, a second user input device 154, and a third user input device 156 are operatively coupled to system bus 102 by user interface adapter 150. The user input devices 152, 154, and 156 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present principles. The user input devices 152, 154, and 156 can be the same type of user input device or different types of user input devices. The user input devices 152, 154, and 156 are used to input and output information to and from system 100.

Of course, the processing system 100 may also include other elements (not shown), as readily contemplated by one of skill in the art, as well as omit certain elements. For example, various other input devices and/or output devices can be included in processing system 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 100 are readily contemplated by one of ordinary skill in the art given the teachings of the present principles provided herein.

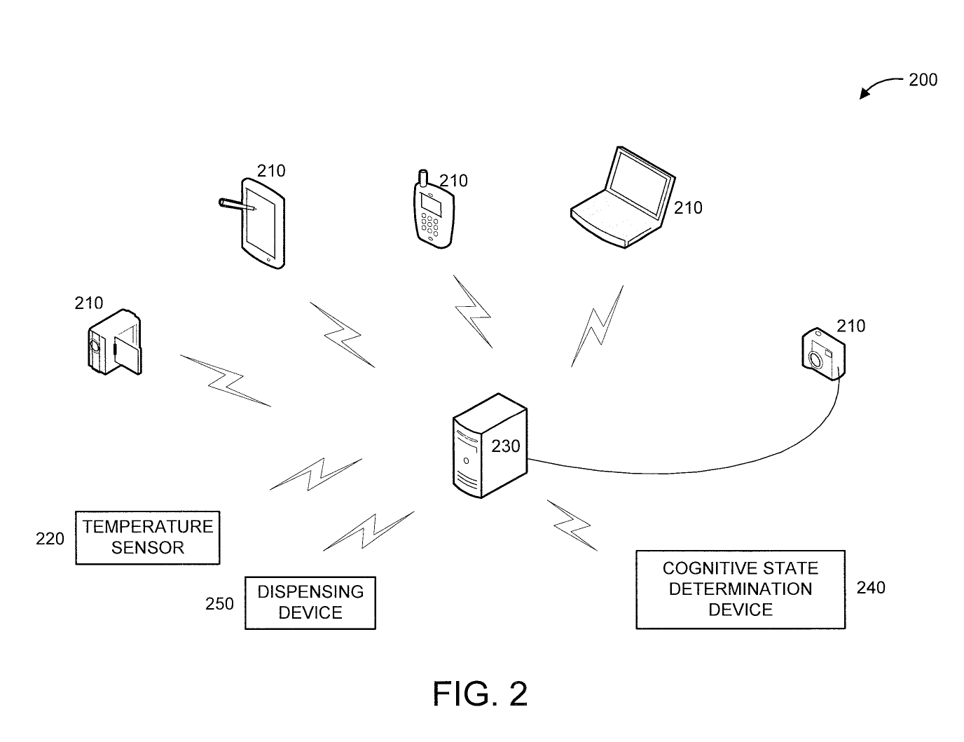

Moreover, it is to be appreciated that system 200 described below with respect to FIG. 2 is a system for implementing respective embodiments of the present principles. Part or all of processing system 100 may be implemented in one or more of the elements of system 200.

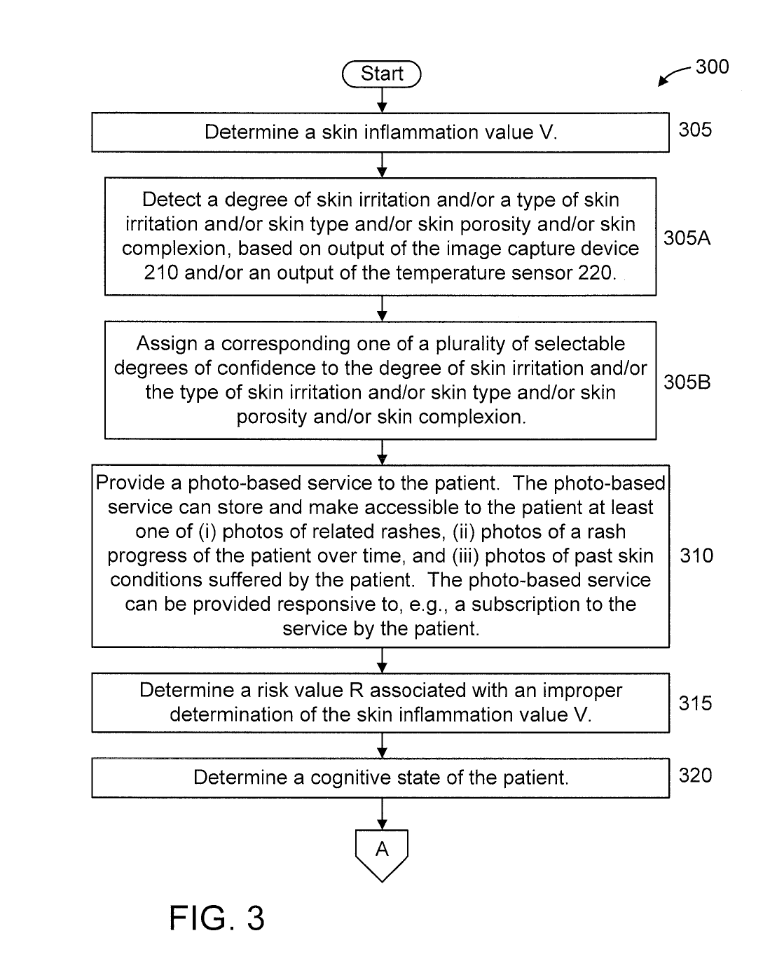

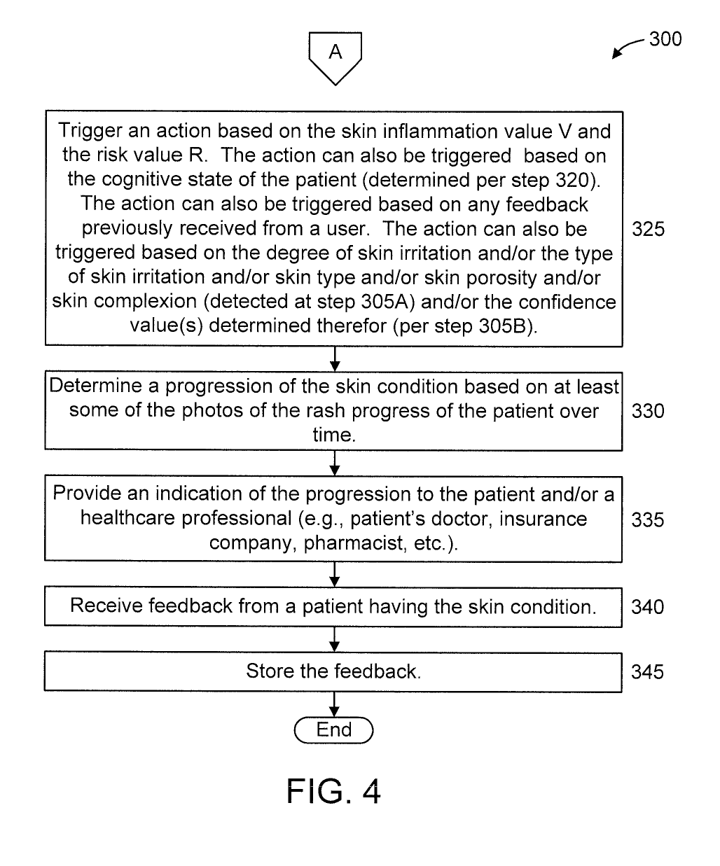

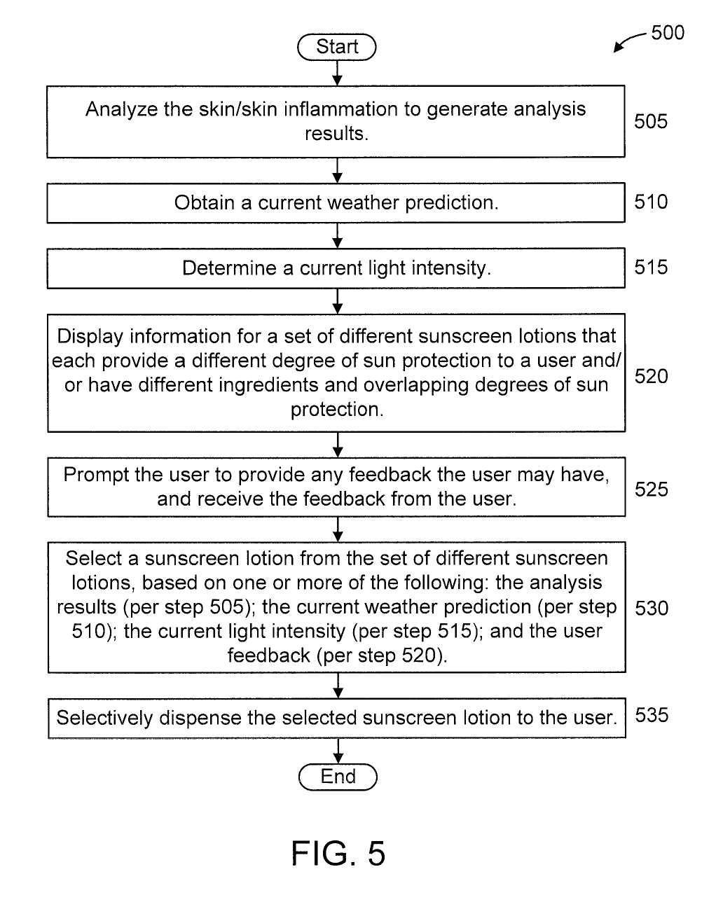

Further, it is to be appreciated that processing system 100 may perform at least part of the method described herein including, for example, at least part of method 300 of FIGS. 3-4 and/or at least part of method 500 of FIG. 5. Similarly, part or all of system 200 may be used to perform at least part of method 300 of FIGS. 3-4 and/or at least part of method 500 of FIG. 5.

FIG. 2 shows a system 200 for skin medicine dose control, in accordance with an embodiment of the present principles.

The system 200 includes an image capture device(s) 210 (hereinafter primarily in singular form for the sake of illustration), a temperature sensor 220, a computer processing system 230, a cognitive state determination device 240, and a dispensing device 250.

The computer processing system 230 can be a standalone computer (e.g., a server) or can be a distributed computer processing system. In an embodiment, part of system 200 can be implemented in the cloud using a cloud configuration. For example, some or all of the functions described herein with respect to computer processing system 230 can be performed in the cloud.

The image capture device 210 and/or the temperature sensor 220 can determine a skin inflammation value V. For example, an elevated local temperature can be indicative of a local infection. In an embodiment, the image capture device 210 is implemented by a camera in a smartphone. Of course, a standalone camera or the camera in other devices (e.g., a media player, e-book, tablet, and so forth) can be used to implement image capture device 210. As such, various different image capture devices 210 are shown, as a particular user can utilize any of the same in accordance with the teachings of the present principles in order to obtain the benefits of the present principles. Some of the image capture devices 210 can involve a wired connection to the computer processing system 230, while other ones of the image capture devices 210 can involve wireless communication with the computer processing system 230.

The computer processing system 230 can detect a degree of skin irritation and/or a type of skin irritation based on output of the image capture device 210 and/or an output of the temperature sensor 220. Moreover, the computer processing system 230 can assign a corresponding one of a plurality of selectable degrees of confidence to the degree of skin irritation and/or the type of skin irritation. In an embodiment, the confidence values can be based on a database of photos showing skin irritations against which photos for a current patient are compared. Moreover, in an embodiment, the confidence values can be based on patient feedback.

The computer processing system 230 can also provide a photo-based service to the patient. For example, in an embodiment, the service can include photos of related rashes, photos of a rash progress of the patient over time, and photos of past skin conditions suffered by the patient. The service can be provided on any of the user's devices including the device that, in turn, includes the image capture device 210, where such device as noted above can be a smartphone, a media player, a tablet, and so forth.

The computer processing device 230 can trigger an action relating to the skin condition based on the skin inflammation value V and the risk value R. Moreover, the action can be triggered also based on the user's cognitive state. Moreover, the action can be triggered also based on feedback provided by the user. Various types of exemplary actions are described below.

The dispensing device 250 can be any type of dispensing device (e.g., any mechanical/electro-mechanical dispensing device including, but not limited to, a pump-spray based device, an aerosol based device, a container, and so forth). In an embodiment, the dispensing device dispenses medicines. In an embodiment, the dispensing device is an automated dispensing device (e.g., one that connects to an intravenous line fed into the patient). In an embodiment, the dispensing device 250 can be controlled by the computer processing system 230. For example, the amount of medicine, the rate of dispensing medicine (e.g., milliliter/hour, etc.), the type of medicine (e.g., in the case of a dispensing device that stores different medicines/compounds that can be selectively dispensed), and so forth can be controlled by the computer processing system 230. In an embodiment, the dispensing device 250 dispenses light, e.g., relating to indoor sunlamp (UV light) therapy, where the duration and intensity of the light exposure is controlled for patients who have skin conditions like Psoriasis. It is to be appreciated that dispensing device 230 can dispense any medical related product, depending upon the implementation, while maintaining the spirit of the present principles. In an embodiment, the dispensing device 230 can be part of a photo-based service, where dispensing is performed responsive to photos or skin inflammation values derived from photos.

The cognitive state determination device 240 determines the cognitive state of the user. In an embodiment, the cognitive state can be determined with respect to any particular targeted cognitive state including, but not limited to, itching; pain; embarrassment; nervousness; discomfort; anxiety; attendance at an event; and so forth. The cognitive state determination device 240 can be implemented using one or more of the following: a camera; a heart rate monitor; a blood pressure monitor; a microphone; a speaker; a calendar; and so forth. The cognitive state determination device 240 can be implemented by wearables. The wearables can include personal wearable instrumentation (e.g., smart watches, blood pressure monitors, and so forth) that measures various parameters of a person. The preceding examples are merely illustrative and, thus, other types of devices can also be used to determine user cognitive state, while maintaining the spirit of the present principles.

Thus, the cognitive state determination device 240 can involve video data/analysis and wearables data/analysis. Moreover, the determination of a user's cognitive state can involve a Hidden Markov Model, a Markov Network, a decision tree, or a set of topological descriptors of graphs constructed by associating these states with nodes and their transitions with edges. Of course, other approaches to recognizing a user's cognitive state can also be used, while maintaining the spirit of the present principles.

Thus, for example, a camera can be used to determine itching (user is scratching), pain (user is wincing from pain, etc.), embarrassment (user has red checks, user is covering arms or other body part where skin condition is visually apparent to others, etc.), predict attendance (user is not moving from his or her home and it's time to leave to the event and/or get ready for the event and/or so forth).

FIGS. 3-4 show a method 300 for skin medicine dose control, in accordance with an embodiment of the present principles. The method is performed with respect to a patient having a skin condition.

At step 305, determine a skin inflammation value V. The skin inflammation value can be determined using, for example, image capture device 210 and/or temperature sensor 220.

In an embodiment, step 305 can involve steps 305A and 305B.

At step 305A, detect a degree of skin irritation (e.g., “little”, “a lot”, etc.) and/or a type of skin irritation (e.g., sunburn, shingles, eczema, etc.) and/or skin type (e.g., “dry”, “moist”, “oily”, etc.) and/or skin porosity (e.g., “small”, “medium”, “large” pores, etc.) and/or skin complexion (e.g., “light”, “medium”, “dark”, etc.), based on output of the image capture device 210 and/or an output of the temperature sensor 220.

At step 305B, assign a corresponding one of a plurality of selectable degrees of confidence to the degree of skin irritation and/or the type of skin irritation and/or skin type and/or skin porosity and/or skin complexion.

At step 310, provide a photo-based service to the patient. The photo-based service can store and make accessible to the patient at least one of (i) photos of related rashes, (ii) photos of a rash progress of the patient over time, and (iii) photos of past skin conditions suffered by the patient. The photo-based service can be provided responsive to, e.g., a subscription to the service by the patient.

At step 315, determine a risk value R associated with an improper determination of the skin inflammation value V. In an embodiment, the risk value R is determined based on patient information (e.g., do not increase the dose beyond a threshold if there is a possible error in the value of the skin inflammation value V and if the patient has been diagnosed with a particular skin condition).

At step 320, determine a cognitive state of the patient. In an embodiment, the cognitive state of the patient is determined with respect to one or more of the following: itching; pain; embarrassment; and predicting attendance of the patient at an event.

At step 325, trigger an action based on the skin inflammation value V and the risk value R. The action can also be triggered based on the cognitive state of the patient (determined per step 320). The action can also be triggered based on any feedback previously received from a user. The action can also be triggered based on the degree of skin irritation and/or the type of skin irritation and/or skin type and/or skin porosity and/or skin complexion (detected at step 305A) and/or the confidence value(s) determined therefore (per step 305B).

Further regarding step 325, the action can be, but is not limited to, one or more of the following: controlling an amount of medication or other medical related product that exits a medicine or other product application device; controlling a protection property (e.g., sun protection factor (SPF) of sunscreen lotion to be applied to a patient having the skin condition (by selecting a sunscreen having a desired level of protection from among multiple sunscreens that offer different levels of protection); sending an alert to a healthcare professional; tracking the skin inflammation value V and the risk value R over time; and deterring a use of a particular medicine in the medicine application device (to determine an inappropriate or potentially harmful treatment). Regarding controlling an amount of medication that exists a medicine application device, the same can be a machine controlled medicine application device, where at least the dosage of the medicine that is administered to a patient is controlled. In an embodiment, the medicine application device can be completely automated (machine controlled), from dosing to actual administering of the medicine (whether by injection, spraying, dripping and/or otherwise providing via an intravenous (IV) device, and so forth). These and other actions to which the present principles can be applied are readily determined by one of ordinary skill in the art given the teachings of the present principles provided herein, while maintaining the spirit of the present principles.

At step 330, determine a progression of the skin condition based on at least some of the photos of the rash progress of the patient over time.

At step 335, provide an indication of the progression to the patient and/or a healthcare professional (e.g., patient's doctor, insurance company, pharmacist, etc.).

At step 340, receive feedback from a patient having the skin condition. The feedback can relate, but is not limited, to pain, swelling, level of discomfort, medicine allergies, and so forth.

At step 345, store the feedback. The feedback can be used when any of the preceding steps are repeated, so that the user's feedback can be considered.

As is appreciated by one of ordinary skill in the art, method 300 can be repeated as needed (for example, when symptoms return), of course, in consideration of any dosing restrictions.

A further description will now be given regarding various steps of method 300, in accordance with various embodiments of the present invention.

Regarding step 305, in an embodiment, an area without inflammation can be considered as baseline with each successive length unit (mm, etc.) of inflammation adding to the baseline inflammation value.

Further regarding step 305, the images and/or temperature readings of a subject user can be compared to the database of images and/or temperature readings (corresponding to the same and/or other users) in order to use the metadata (temperature, inflammation value) associated with the images and/or temperature readings in the database. Thus, when a currently taken image or currently taken temperature reading matches an image or temperature reading in the database, then the metadata associated with the image and/or temperature reading in the database is also associated with the currently taken image and/or temperature reading in order to label the currently taken image and/or temperature reading.

Additionally regarding step 305, the skin inflammation value can be determined from one or more labels that have been determined from evaluating an image of a user. For example, the following labels can be assigned for the following determinations):
(a) degree of skin irritation (e.g., “little”, “a lot”, etc.); and/or
(b) type of skin irritation (e.g., sunburn, shingles, eczema, etc.); and/or
(c) skin type (e.g., “dry”, “moist”, “oily”, etc.); and/or
(d) skin porosity (e.g., “small”, “medium”, “large” pores, etc.); and/or
(e) skin complexion (e.g., “light”, “medium”, “dark”, etc.).
   where these labels are then processed to determine a skin inflammation value, as would be readily appreciated by one of ordinary skill in the art.

The labels can be obtained by comparing currently taken images of a user to a database of images (of the same and/or other users), and assigning the same label to the currently taken images as those assigned to the images in the database when a match exists there between.

In an embodiment, the degree of skin irritation can be indicated using labels, a respective numerical value, a respective numerical range and/or so forth, depending upon the implementation.

Hence, in an embodiment, even the user him or herself can serve as a baseline (where, e.g., a previous image showing no inflammation has an inflammation value of zero or a previous local temperature reading showing no increased local temperature (i.e., the person and the local region in issue have the same temperature) also has an inflammation value of zero) and where each darker shade of red captured in subsequent images or each higher degree of local temperature increases the corresponding value of the inflammation value in a range (which can arbitrarily be from 1-10, 1-100, A-D, and so forth, as the convention used is not important, only consistency in its application as would be readily apparent to one of ordinary skill in the art). In this way, V and R can be tracked through time (e.g., in a photo log formed for the user, e.g., in step 310).

Regarding step 305B, the confidence values are different from the risk value, as clearly shown and described with respect to elements 305B and 315 of FIG. 3. In particular, element 305B assigns a corresponding one of a plurality of selectable degrees of confidence (how correct) and element 315 determines a risk value R. In an embodiment, the confidence values can be based on a database of photos showing skin irritations against which photos for a current patient are compared. As an example, if a currently taken image matches 10 images in a database relating to one (first) skin inflammation value, and 1 image in the database relating to another (second) skin inflammation value, then the confidence value can be 10 in relation to the first skin inflammation value and 1 in relation to the second skin inflammation value. Or, in the case of 100 images in the database, the confidence can be 10% for the first skin inflammation value and 1% for the second skin inflammation value. Other approaches can also be used. Moreover, in an embodiment, the confidence values can be based on patient feedback. For example, a patient can themselves assign a corresponding confidence value. Additionally, the metadata for the photos in the database can have confidence values associated therewith, or the confidence values can be based on matching a current photo to one in the database. The confidence values can be based on the metadata associated with the photos in the database. These and other variations are readily determined by one of ordinary skill in the art given the teachings of the present invention provided herein.

Regarding step 315, risk value R can be based on a particular skin condition diagnosis for a user, a particular medicine and/or corresponding dosage thereof taken by the user, and so forth. Hence, as an example readily apparent to one of ordinary skill in the art given the teachings of the present invention provided in the instant specification as filed, skin cancer patients may have a higher risk value assigned than patients with Eczema. Thus, risk values can be simply “binned” so to speak based on the corresponding skin condition diagnosis such that potentially fatal skin conditions receive a certain high risk value, while likely non-fatal skin conditions receive a certain lower risk value, and so on. Hence, the risk value can be based on a danger level, whereas a confidence level is based on a degree of correctness.

In an embodiment, the risk value is associated with an improper determination of the skin inflammation value, and which, in turn, can be obtained from patient information. That is, the risk value is associated with improperly determining the skin inflammation value. Thus, in an embodiment, from patient information where it is determined that the underlying skin condition (patient information) is acne, the risk value would be relatively low as compared to the underlying skin condition being skin cancer. It is from type of reasoning as an example, that the risk value can be ascertained. In an embodiment, the risk value can represent a degree of danger in an improper determination of skin inflammation value. Clearly, the risk for a mis-determination of acne, sunburn, etc. is smaller than a mis-determination of cancer with respect to the danger faced by the patient. Family history can also be considered as relevant patient information for determining the risk value. Hence, the risk value would increase with increasing numbers of family members who have had a particular skin condition. These and other risk factors can be used in determining the risk value.

In an embodiment, patient information can be information particularly related to a user's underlying skin condition, their medicines and corresponding dosages for the skin conditions, their family history in relation to this and related skin conditions, and so forth. Thus, patient information is information relating to a patient and one or more potential skin conditions. The user's eating schedule would thus not be patient information in this context, but a person's sunning schedule on vacation would be patient information in this context. This and other examples of the same are readily determined by one of ordinary skill in the art, given the teachings of the present invention provided herein.

FIG. 5 shows another method 500 for skin medicine dose control, in accordance with an embodiment of the present principles. Part or all of method 500 can be integrated with method 300, as readily appreciated by one of ordinary skill in the art given the teachings of the present principles provided herein, while maintaining the spirit of the present principles. It is to be appreciated that while method 500 is described with respect to sunscreen lotions for the sake of illustration, method 500 can be readily applied to any type of medicine to provide skin medicine dose control. These and other variations and aspects of the present principles are readily determined by one of ordinary skill in the art given the teachings of the present principles provided herein, while maintaining the spirit of the present principles.

At step 505, analyze the skin/skin inflammation to generate analysis results. For method 500, this step can be performed, for example, for a person who has a current skin condition and nonetheless plans to spend some time exposed to the sun. If the person does not currently have a skin condition, then this step can be skipped, and step 530 would not consider the results of the analysis but would consider the other mentioned items. In an embodiment, the analysis results can include the inflammation value V described with respect to step 305 of method 300 of FIG. 3. In an embodiment, the analysis results can include a degree of skin irritation and/or a type of skin irritation and/or a skin type and/or a skin porosity and/or a skin complexion.

At step 510, obtain a current weather prediction. The weather prediction can include temperature, amount of sun (e.g., sun index)), amount of cloud cover, amount of wind and so forth.

The weather prediction can be obtained by the transceiver 142 of FIG. 1 and/or image capture device 210 of FIG. 2. Weather prediction often state cloudy, sunny, overcast, etc., allowing for ascertaining, to some degree, the light intensity. In the case of the camera, the light intensity can be more readily and accurately determined as a normal camera output, as readily appreciated by one of ordinary skill in the art. In fact, many cameras have a user ascertainable element, usually an icon, indicative of the light intensity of a picture.

At step 515, determine a current light intensity.

At step 520, display information for a set of different sunscreen lotions that each provide a different degree of sun protection to a user and/or have different ingredients and overlapping degrees of sun protection. For example, regarding the latter, one sunscreen may provide the same protection as another, but have different ingredients (as an ingredient may be the cause of the skin inflammation).

At step 525, prompt the user to provide any feedback the user may have, and receive the feedback from the user. For example, the user may be allergic to a particular type of sunscreen based on its ingredients and may rule that one out from consideration/application in their feedback. The feedback can also include the time of day (e.g., midday, 1-4 PM, etc.) the user plans on being exposed to the sun, and the duration of the exposure.

At step 530, select a sunscreen lotion from the set of different sunscreen lotions, based on one or more of the following: the analysis results (per step 505); the current weather prediction (per step 510); the current light intensity (per step 515); and the user feedback (per step 520).

At step 535, selectively dispense the selected sunscreen lotion to the user. In an embodiment, step 535 involves spraying the user with the selected sunscreen lotion. In an embodiment, the step of selectively dispensing a particular sunscreen lotion from a set of different sunscreen lotions can be considered the action triggered in step 325 of method 300 of FIG. 3.

In an embodiment, at least the analyzing (step 505), selecting (step 530), and dispensing (step 535) steps are provided by a skin care service (see, e.g., step 310 of method 300) to which the patient having the skin condition subscribes. The service can involve maintaining data for users in a database, allowing users to retrieve images and/or temperature values and/or other values such as skin inflammation and/or risk values. The service can prompt the user to take a picture in order to compare it to other images for purpose of classifying the user regarding, e.g., skin inflammation value and/or so forth. The service can store the pictures that it prompted the user to take and can form a photo library of a particular outbreak of the user. In an embodiment, the service can provide information to a user that describes their skin condition, where the information is determined based on the skin inflammation value and/or other information (e.g., a health care provider's diagnosis, etc.). In an embodiment, the skin care service is a service provided to a user that provides one or more services directed to at least a skin inflammation value of a user and/or a diagnosed skin condition of the user. For example, the skin care service can involve dispensing indoor Sunlamp (UV light) therapy by controlling the duration and intensity of the light exposure for patients who have skin conditions like Psoriasis. Hence, the skin care service can be a service for curing and/or otherwise treating a skin condition and/or skin inflammation value.

As is appreciated by one of ordinary skill in the art, method 500 can be repeated as needed (for example, when symptoms (e.g., of burning) return, after swimming where the efficacy of the sunscreen lotion is likely diminished, and so forth), of course, in consideration of any dosing restrictions.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based email). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load balancing between clouds).

A cloud computing environment is service oriented with a focus on statelessness, low coupling, modularity, and semantic interoperability. At the heart of cloud computing is an infrastructure comprising a network of interconnected nodes.

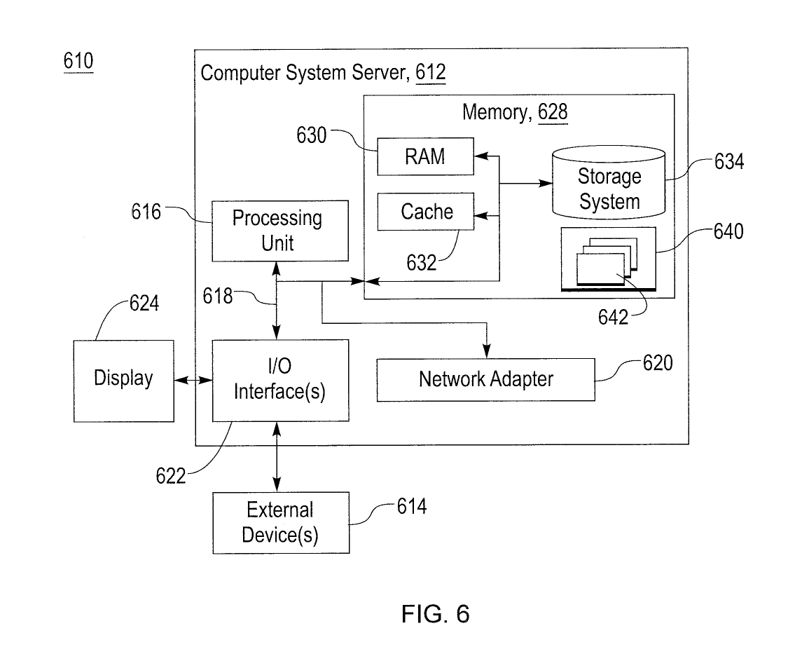

Referring now to FIG. 6, a schematic of an example of a cloud computing node 610 is shown. Cloud computing node 610 is only one example of a suitable cloud computing node and is not intended to suggest any limitation as to the scope of use or functionality of embodiments of the invention described herein. Regardless, cloud computing node 610 is capable of being implemented and/or performing any of the functionality set forth hereinabove.

In cloud computing node 610 there is a computer system/server 612, which is operational with numerous other general purpose or special purpose computing system environments or configurations. Examples of well-known computing systems, environments, and/or configurations that may be suitable for use with computer system/server 612 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 612 may be described in the general context of computer system executable instructions, such as program modules, being executed by a computer system. Generally, program modules may include routines, programs, objects, components, logic, data structures, and so on that perform particular tasks or implement particular abstract data types. Computer system/server 612 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 612 in cloud computing node 610 is shown in the form of a general-purpose computing device. The components of computer system/server 612 may include, but are not limited to, one or more processors or processing units 616, a system memory 628, and a bus 618 that couples various system components including system memory 628 to processor 616.

Bus 618 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 612 typically includes a variety of computer system readable media. Such media may be any available media that is accessible by computer system/server 612, and it includes both volatile and non-volatile media, removable and non-removable media.

System memory 628 can include computer system readable media in the form of volatile memory, such as random access memory (RAM) 630 and/or cache memory 632. Computer system/server 612 may further include other removable/non-removable, volatile/non-volatile computer system storage media. By way of example only, storage system 634 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 618 by one or more data media interfaces. As will be further depicted and described below, memory 628 may include at least one program product having a set (e.g., at least one) of program modules that are configured to carry out the functions of embodiments of the invention.

Program/utility 640, having a set (at least one) of program modules 642, may be stored in memory 628 by way of example, and not limitation, as well as an operating system, one or more application programs, other program modules, and program data. Each of the operating system, one or more application programs, other program modules, and program data or some combination thereof, may include an implementation of a networking environment. Program modules 642 generally carry out the functions and/or methodologies of embodiments of the invention as described herein.

Computer system/server 612 may also communicate with one or more external devices 614 such as a keyboard, a pointing device, a display 624, etc.; one or more devices that enable a user to interact with computer system/server 612; and/or any devices (e.g., network card, modem, etc.) that enable computer system/server 612 to communicate with one or more other computing devices. Such communication can occur via Input/Output (I/O) interfaces 622. Still yet, computer system/server 612 can communicate with one or more networks such as a local area network (LAN), a general wide area network (WAN), and/or a public network (e.g., the Internet) via network adapter 620. As depicted, network adapter 620 communicates with the other components of computer system/server 612 via bus 618. It should be understood that although not shown, other hardware and/or software components could be used in conjunction with computer system/server 612. Examples, include, but are not limited to: microcode, device drivers, redundant processing units, external disk drive arrays, RAID systems, tape drives, and data archival storage systems, etc.

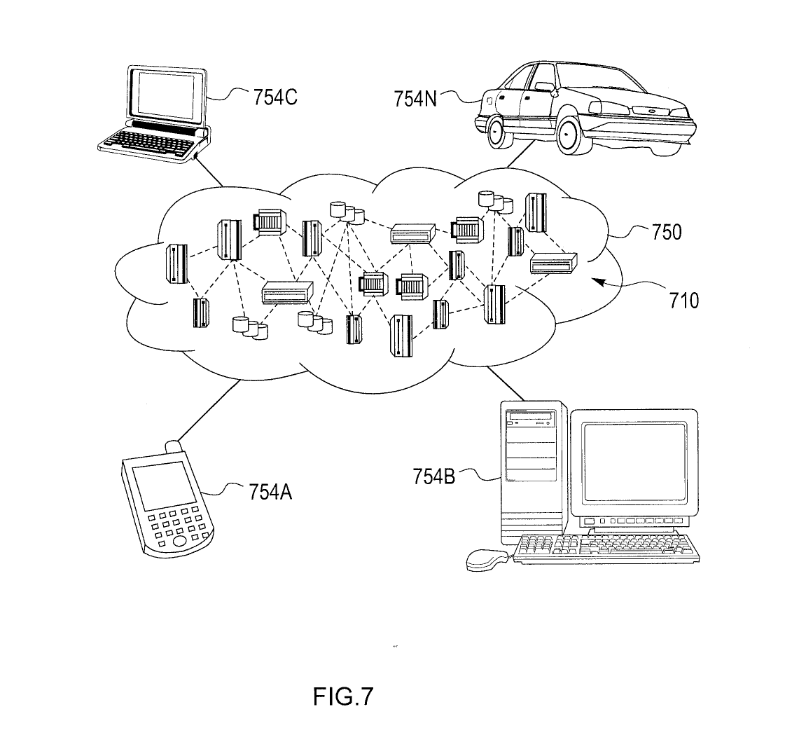

Referring now to FIG. 7, illustrative cloud computing environment 750 is depicted. As shown, cloud computing environment 750 comprises one or more cloud computing nodes 710 with which local computing devices used by cloud consumers, such as, for example, personal digital assistant (PDA) or cellular telephone 754A, desktop computer 754B, laptop computer 754C, and/or automobile computer system 754N may communicate. Nodes 710 may communicate with one another. They may be grouped (not shown) physically or virtually, in one or more networks, such as Private, Community, Public, or Hybrid clouds as described hereinabove, or a combination thereof. This allows cloud computing environment 750 to offer infrastructure, platforms and/or software as services for which a cloud consumer does not need to maintain resources on a local computing device. It is understood that the types of computing devices 754A-N shown in FIG. 7 are intended to be illustrative only and that computing nodes 710 and cloud computing environment 750 can communicate with any type of computerized device over any type of network and/or network addressable connection (e.g., using a web browser).

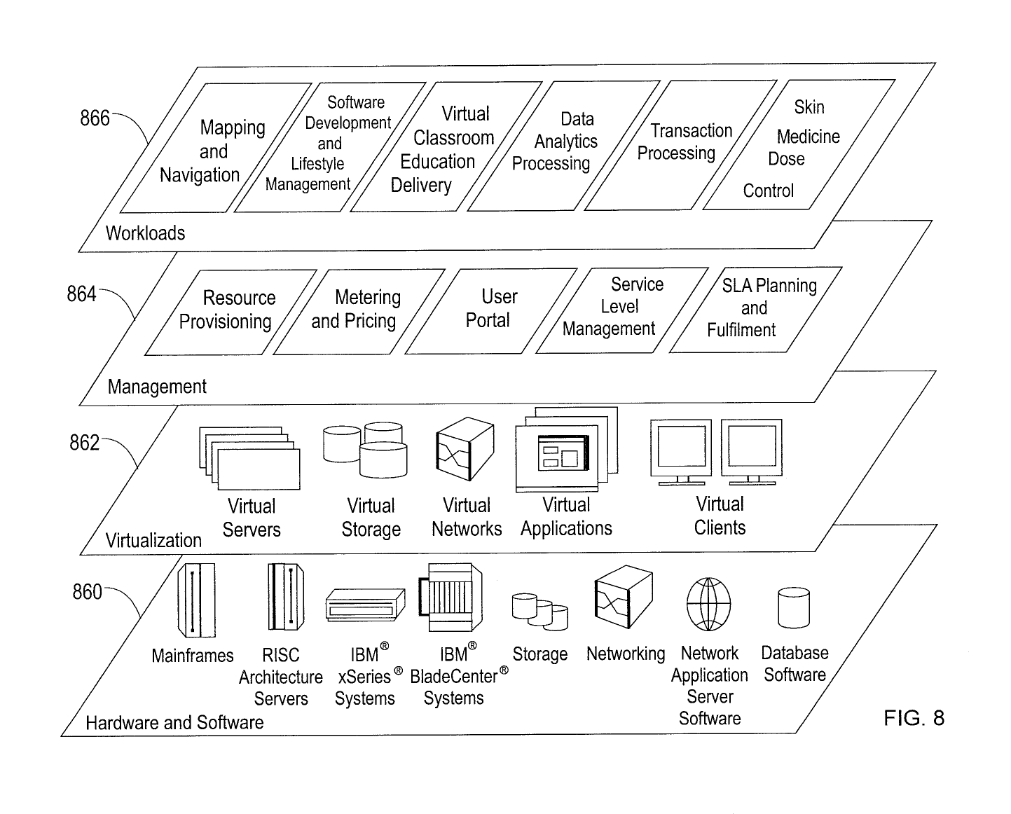

Referring now to FIG. 8, a set of functional abstraction layers provided by cloud computing environment 750 (FIG. 7) is shown. It should be understood in advance that the components, layers, and functions shown in FIG. 8 are intended to be illustrative only and embodiments of the invention are not limited thereto. As depicted, the following layers and corresponding functions are provided:

Hardware and software layer 860 includes hardware and software components. Examples of hardware components include mainframes; RISC (Reduced Instruction Set Computer) architecture based servers; blade server systems; storage devices; networks and networking components. Examples of software components include network application server software; and database software.

Virtualization layer 862 provides an abstraction layer from which the following examples of virtual entities may be provided: virtual servers; virtual storage; virtual networks, including virtual private networks; virtual applications and operating systems; and virtual clients.

In one example, management layer 864 may provide the functions described below. Resource provisioning provides dynamic procurement of computing resources and other resources that are utilized to perform tasks within the cloud computing environment. Metering and Pricing provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal provides access to the cloud computing environment for consumers and system administrators. Service level management provides cloud computing resource allocation and management such that required service levels are met. Service Level Agreement (SLA) planning and fulfillment provide pre-arrangement for, and procurement of, cloud computing resources for which a future requirement is anticipated in accordance with an SLA.

Workloads layer 866 provides examples of functionality for which the cloud computing environment may be utilized. Examples of workloads and functions which may be provided from this layer include: mapping and navigation; software development and lifecycle management; virtual classroom education delivery; data analytics processing; transaction processing; and skin medicine dose control.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A method for managing a skin condition, the method comprising:
determining, using at least one of an image capture device and a temperature sensor, a skin inflammation value;
determining, by a hardware processor, a risk value associated with an improper determination of the skin inflammation value; and
triggering, by the hardware processor, an action relating to the skin condition based on the skin inflammation value and the risk value.

2. The method of claim 1, wherein the action comprises controlling an amount or a rate of medication that exits a medicine application device.

3. The method of claim 1, wherein the action comprises controlling a protection property of sunscreen lotion to be applied to a patient having the skin condition.

4. The method of claim 1, wherein the action comprises at least one of sending an alert to a healthcare professional, tracking the skin inflammation value and the risk value over time, and deterring a use of a particular medicine in the medicine application device.

5. The method of claim 1, wherein determining the risk value is based on patient information.

6. The method of claim 1, further comprising receiving, by the hardware processor, feedback from a patient having the skin condition, and wherein said triggering step is further based on the feedback.

7. The method of claim 1, further comprising providing, by a cloud server operatively coupled to the hardware processor, a photo-based service to a patient having the skin condition, wherein the photo-based service stores and makes accessible to the patient at least one of (i) photos of related rashes, (ii) photos of a rash progress of the patient over time, and (iii) photos of past skin conditions suffered by the patient.

8. The method of claim 7, further comprising determining, by the hardware processor, a progression of the skin condition based on at least some of the photos of the rash progress of the patient over time.

9. The method of claim 1, wherein the image capture device that determines the skin inflammation value is comprised in a smartphone.

10. The method of claim 1, further comprising determining a cognitive state of a patient having the skin condition, and wherein said triggering step is further based on the cognitive state of the patient.

11. The method of claim 1, wherein the cognitive state of the patient is determined with respect to at least one of itching, pain, embarrassment, and predicting attendance of the patient at an event.

12. The method of claim 1, further comprising detecting, by the hardware processor, at least one of a degree of skin irritation and a type of skin irritation based on an output of the temperature sensor.

13. The method of claim 12, wherein said detecting step comprises assigning a corresponding one of a plurality of selectable degrees of confidence to the at least one of the degree of skin irritation and the type of skin irritation.

14. The method of claim 1, further comprising selectively dispensing, by a dispensing apparatus as the triggered action, one of a plurality of sunscreen lotions, each providing a different degree of sun protection.

15. The method of claim 14, further comprising analyzing a skin inflammation to generate analysis results, and selecting the one of the plurality of sunscreen lotions based on the analysis results.

16. The method of claim 15, wherein at least said dispensing, analyzing, and selecting steps are provided by a skin care service to which the patient having the skin condition subscribes.

17. The method of claim 15, further comprising providing, on a display device, information on at least some of the plurality of sunscreen lotions.

18. The method of claim 15, further comprising obtaining a weather prediction and determining a current light intensity, and wherein said selecting step is further based on the weather prediction and the current light intensity.

19. A non-transitory computer readable storage medium comprising a computer readable program for managing a skin condition, wherein the computer readable program when executed on a computer causes the computer to perform the steps of:
determining, using at least one of an image capture device and a temperature sensor, a skin inflammation value;
determining, by a hardware processor, a risk value associated with an improper determination of the skin inflammation value; and
triggering, by the hardware processor, an action relating to the skin condition based on the skin inflammation value and the risk value.

20. A system, comprising:
at least one of an image capture device and a temperature sensor for determining a skin inflammation value; and
a hardware processor for determining a risk value associated with an improper determination of the skin inflammation value, and triggering an action relating to the skin condition based on the skin inflammation value and the risk value.
